(19) United States
(12) Patent Application Publication
(10) Pub. No.: US 2024/0207151 A1
(43) Pub. Date: Jun. 27, 2024

(54) LIQUID COMPOSITION

(71) Applicant: SHISEIDO COMPANY, LTD., Chuo-ku, Tokyo (JP)

(72) Inventors: Shiho IWAMI, Tokyo (JP); Kouichi NAGAI, Tokyo (JP); Kei UJIMOTO, Tokyo (JP); Ryoya ITO, Tokyo (JP)

(73) Assignee: SHISEIDO COMPANY, LTD., Chuo-ku, Tokyo (JP)

(21) Appl. No.: 18/557,674

(22) PCT Filed: Mar. 23, 2022
(86) PCT No.: PCT/JP2022/013642

§ 371 Date: Oct. 27, 2023

(30) Foreign Application Priority Data
May 28, 2021 (JP) 2021-090643

(51) Int. Cl. A61K 8/27; A61K 8/19; A61K 8/29; A61Q 17/04; C09C 1/04; C09C 3/12

(52) U.S. Cl. CPC A61K 8/27; A61K 8/19; A61K 8/29; A61Q 17/04; C09C 1/043; C09C 3/12; A61K 2800/412; A61K 2800/413; A61K 2800/434; A61K 2800/612; A61K 2800/614; C01P 2004/61

(57) ABSTRACT

To a novel liquid composition comprising C₅₋₁₀ alkyl tri-C₁₋₃ alkoxysilane-treated zinc oxide particles, and metal compound particles.

Redispersibility of metal compound particles in a liquid composition is improved.

A liquid composition comprising C₅₋₁₀ alkyl tri-C₁₋₃ alkoxysilane-treated zinc oxide particles, and metal compound particles.

### FIELD

The present invention relates to a liquid composition and a redispersion facilitator.

### BACKGROUND

Metal compound particles contained in cosmetics, paints, and the like are used as raw materials having various functions. In particular, these metal compound particles have the characteristic that various effects can be obtained depending on the size (average particle diameter) thereof. Metal compound particles having a large average particle diameter may be used as white pigments as colorants, those having a small average particle diameter have a function in that they are excellent in UV reduction effect, and doped metal compound particles function as phosphors and are used in sunscreen cosmetics and the like.

Zinc oxide particles scatter both UVA and UVB rays and are thus used as UV scattering agents in ointments, creams, and lotions to protect against sunburn and other UV-induced skin damage. Furthermore, some zinc oxide particles can be phosphors which are each used in cosmetics and the like.

Magnesium titanate particles are doped with manganese to form phosphors which are used in cosmetics and the like (Patent Literature 1).

Titanium mica, which consists of a base material of muscovite coated thereon with a thin layer of titanium oxide, has colored reflective interference colors such as red, yellow, blue, and green, and is conventionally used in cosmetics as a pearlescent agent.

Bismuth oxychloride (BiOCl) has a unique soft luster and is non-toxic and is thus used as a pigment blended in various cosmetics such as makeup cosmetics and nail products.

Barium sulfate is used as an additive for cosmetics such as skin creams and sunscreens.

Calcium cerium phosphate (Ca₄P₂O₉:Ce) consisting of calcium phosphate partially doped with cerium is known as a phosphor (Patent Document 2) and used not only as a base material for cosmetic phosphor materials, but also for a wide variety of other purposes, such as suppressing makeup deterioration due to sebum adsorption and achieving a soft focus effect due to light scattering.

Metal compound particles having a large specific gravity and a large particle size may precipitate in a liquid composition to form agglomerates. In order to maintain the dispersibility of metal compound particles, suppress the formation of aggregates, and improve redispersibility, the particle surface may be modified (sometimes called “surface-treated” or “coated”) or a dispersant may be used. For example, in order to disperse zinc oxide of 100 nm or more, a dispersant such as polyhydroxystearic acid is used (Patent Literature 3). Coated barium sulfate imparts advantageous rheological properties to the application matrix and is redispersible in solvents in adhesives, dyes, or cosmetics (Patent Literature 4). Further, in order to improve the redispersibility of titanium mica in hair cosmetics, a carboxyvinyl polymer and an amphoteric polymer are used in combination (Patent Literature 5).

Alkylalkoxysilane is used for surface treatment (hydrophobic treatment) of metal compound particles, and triethoxyoctyl zinc oxide, which functions as an ultraviolet scattering agent, is included, for example, in makeup bases (Patent Literature 6).

### CITATION LIST

### Patent Literature

[PTL 1] WO 2016/017372
[PTL 2] Japanese Unexamined Patent Publication (Kokai) No. 2020-50733
[PTL 3] Japanese Unexamined PCT Publication (Kohyo) No. 2012-511499
[PTL 4] Japanese Unexamined PCT Publication (Kohyo) No. 2003-535010
[PTL 5] Japanese Unexamined Patent Publication (Kokai) HEI No. 11-171729
[PTL 6] Japanese Unexamined Patent Publication (Kokai) No. 2017-088599

### SUMMARY

### Technical Problem

However, the redispersibility of agglomerates produced by the precipitation of metal compound particles in a liquid composition is not sufficient even with coating or the addition of a dispersant. Significant effort is required in order to stir (or rotate) and redisperse the metal compound particles generated in the liquid composition, and if the liquid composition without sufficient redispersion is used for a long time, the component composition could become inhomogeneous, leading to a failure to bring about the expected effects of the liquid composition or deterioration of stability during storage.

### Solution to Problem

Under such circumstances, the present inventors have extensively studied a third component for further improving the redispersibility of metal compound particles which tend to agglomerate. As a result, it was discovered that C₅₋₁₀ alkyl tri-C₁₋₃ alkoxysilane-treated zinc oxide, which is commonly used as an ultraviolet scattering agent, functions as a redispersion facilitator for metal compound particles, and there was discovered a liquid composition having excellent redispersibility, comprising metal compound particles and C₅₋₁₀ alkyl tri-C₁₋₃ alkoxysilane-treated zinc oxide.

Specifically, the present invention provides:

(1) A liquid composition, comprising:
(A) C₅₋₁₀ alkyl tri-C₁₋₃ alkoxysilane-treated zinc oxide particles having an average particle diameter of 100 nm or less, and
(B) metal compound particles having a concentration of 0.2 wt % or more, a specific gravity of 3 or more, and an average particle diameter of 1 μm or more.
   (2) The liquid composition according to (1), wherein a concentration of the C₅₋₁₀ alkyl tri-C₁₋₃ alkoxysilane-treated zinc oxide particles is 1 wt % or more.
   (3) The liquid composition according to (1) or (2), wherein a viscosity of the liquid composition is 10000 mPa·s or less.
   (4) The liquid composition according to any of (1) to (3), wherein the metal compound particles are one or more powders selected from the group consisting of barium sulfate, titanium mica, zinc oxide, zinc oxide phosphor, magnesium titanate, magnesium titanate phosphor, calcium phosphate, calcium cerium phosphate phosphor, and bismuth oxychloride.
   (5) The liquid composition according to any of (1) to (4), wherein the C₅₋₁₀ alkyl tri-C₁₋₃ alkoxysilane-treated zinc oxide particles are octyltriethoxysilane-treated zinc oxide particles or octyltrimethoxysilane-treated zinc oxide particles.
   (6) The liquid composition according to any of (1) to (5), wherein the metal compound particles, when precipitated, are redispersible.
   (7) The liquid composition according to any of (1) to (6), which is a water-in-oil composition.
   (8) The liquid composition according to any of (1) to (7), which is a sunscreen cosmetic.
   (9) A redispersion facilitator for metal compound particles which precipitate and agglomerate in a liquid composition, the redispersion facilitator comprising C₅₋₁₀ alkyl tri-C₁₋₃ alkoxysilane-treated zinc oxide particles as an active ingredient.
   (10) The redispersion facilitator according to (9), wherein the metal compound particles are a powder having a specific gravity of 3 or more and an average particle diameter of 1 μm or more.
   (11) The redispersion facilitator according to (9) or (10), wherein the metal compound particles are one or more powders selected from the group consisting of barium sulfate, titanium mica, zinc oxide, zinc oxide phosphor, magnesium titanate, magnesium titanate phosphor, calcium phosphate, calcium cerium phosphate phosphor, and bismuth oxychloride.
   (12) The redispersion facilitator according to any of (9) to (11), wherein the C₅₋₁₀ alkyl tri-C₁₋₃ alkoxysilane-treated zinc oxide particles are octyltriethoxysilane-treated zinc oxide particles or octyltrimethoxysilane-treated zinc oxide particles.
   (13) Use of the redispersion facilitator according to any of (9) to (12), for redispersion of metal compound particles which precipitate and agglomerate in a liquid composition.

### Advantageous Effects of Invention

The C₅₋₁₀ alkyl tri-C₁₋₃ alkoxysilane-treated zinc oxide according to the present invention is suitable for the redispersion of precipitated metal compound particles in a liquid composition. Since the liquid composition of the present invention containing metal compound particles and C₅₋₁₀ alkyl tri-C₁₋₃ alkoxysilane-treated zinc oxide has excellent redispersibility, it can be used immediately because it requires less labor such as stirring (or rotation) during use, and it is also suitable for long-term use because there is little variation in the composition of ingredients due to long-term use.

### DESCRIPTION OF EMBODIMENTS

The specific embodiments of the present invention will be described in detail below: However, the present invention is not bound by the following embodiments, and arbitrary forms can be adopted within a scope which does not deviate from the spirit of the present invention.

Note that all patent publications, patent application publications, and non-patent literature cited in the present disclosure are incorporated by reference in their entirety into the present disclosure for all purposes.

In the present disclosure, the term “to” when used with numerical values refers to a range of values that are greater than or equal to a specified reference value and less than or equal to a specified reference value.

### (A) C₅₋₁₀ alkyl tri-C₁₋₃ alkoxysilane-Treated Zinc Oxide Particles

The C₅₋₁₀ alkyl tri-C₁₋₃ alkoxysilane of the C₅₋₁₀ alkyl tri-C₁₋₃ alkoxysilane-treated zinc oxide particles used in the present invention is a silane compound having an alkyl group having 5 to 10 carbon atoms and three alkoxy groups having 1 to 3 carbon atoms, and is reactive with zinc oxide compounds. Such silane compounds are represented by the following general formula (1).

RSiX₃ (1)

where R represents an alkyl group having 5 to 10 carbon atoms (which may be linear or branched), and each X independently represents an alkoxy group having 1 to 3 carbon atoms.

In general formula (1) above, the alkyl group represented by R is an alkyl group having 5 to 10 carbon atoms, and examples thereof include a pentyl group, hexyl group, heptyl group, octyl group, nonyl group, and decyl group, which may be a linear or branched chain. Examples of the alkoxy group represented by X in general formula (1) above include alkoxy groups having 1 to 3 carbon atoms such as a methoxy group, ethoxy group, propoxy group, and isopropoxy group.

Specific examples of silane compounds include pentyltrimethoxysilane, hexyltrimethoxysilane, heptyltrimethoxysilane, octyltrimethoxysilane, nonyltrimethoxysilane, decyltrimethoxysilane, pentyltriethoxysilane, hexyltriethoxysilane, heptyltriethoxysilane, octyltriethoxysilane, nonyltriethoxysilane, decyltriethoxysilane, pentyltripropoxysilane, hexyltripropoxysilane, heptyltripropoxysilane, octyltripropoxysilane, nonyltripropoxysilane, decyltripropoxysilane, pentyltriisopropoxysilane, hexyltriisopropoxysilane, heptyltriisopropoxysilane, octyltriisopropoxysilane, nonyltriisopropoxysilane, and decyltriisopropoxysilane. Among these, octyltriethoxysilane and octyltrimethoxysilane are particularly preferred. The silane compound is characterized by easy uniform treatment, easy supply, and inexpensive cost, and zinc oxide particles surface-treated with these compounds have excellent redispersibility characteristics of the metal compound particles contained in the liquid composition of the present invention.

Examples of the method for treating zinc oxide particles with the silane compound include a method of a chemical reaction of the silane compound via reactive groups (alkoxy group, etc.) on the surface of the zinc oxide particle powder, such as a method, in which the silane compound and the zinc oxide particles (powder) are mixed in an organic solvent such as n-hexane, cyclohexane, or a lower alcohol, and the powder is optionally pulverized, and thereafter, the organic solvent is removed by heating or pressure reduction, and heat treatment is carried out, preferably at 80 to 250° C.

Another example is the method described in Japanese Unexamined Patent Publication (Kokai) No. 2007-326902, in which a zinc oxide particle powder is coated with a specific polysiloxane compound and the surface thereof is then treated with the silane compound or a silazane compound in water.

As another example, there is a method in which a zinc oxide particle powder is coated thereon in advance with an inorganic oxide such as silica, alumina, zirconia, titanium oxide, iron oxide, or cerium oxide, and the inorganic oxide-treated zinc oxide particle powder is then surface-coated with the silane compound. Examples of a method for producing the inorganic oxide-treated zinc oxide particle powder include conventionally-known treatment methods such as wet treatment methods using a solvent and mechanochemical methods. An example is the method described in WO 98/17730, in which a zinc oxide particle powder is coated thereon with a silicone compound and then fired to obtain a silica-coated zinc oxide particle powder.

The coating amount of the silane compound on the zinc oxide particle powder is preferably 3 to 15 mass%, and more preferably 4 to 10 mass %, based on the total amount of the zinc oxide particle powder used. Within this range, the zinc oxide particle powder is uniformly surface-coated with the silane compound, whereby there is no agglomeration or precipitation of the silane compound on the surface of the zinc oxide particle powder.

The C₅₋₁₀ alkyl tri-C₁₋₃ alkoxysilane-treated zinc oxide particles of the present invention preferably have an average particle diameter of 200 nm or less, 100 nm or less, 50 nm or less, or 40 nm or less, and more preferably 50 nm or less, 40 nm or less, or 35 nm or less. The average particle diameter of the C₅₋₁₀ alkyl tri-C₁₋₃ alkoxysilane-treated zinc oxide particles is preferably less than the average particle diameter of the metal compound, and is, for example, 1/10 or less, 1/20 or less, 1/30 or less, 1/40 or less, 1/50 or less, 1/90 or less, 1/100 or less, 1/150 or less, or 1/200 or less of the average particle diameter of the metal compound (before agglomeration). Within this range, the redispersibility characteristics are excellent when the metal compound particles contained in the liquid composition of the present invention agglomerate and precipitate.

The C₅₋₁₀ alkyl tri-C₁₋₃ alkoxysilane-treated zinc oxide particles used in the present invention are, relative to the entire liquid composition, 0.1 wt % or more, 0.5 wt % or more, 1.0 wt % or more, 3 wt % or more, 5 wt % or more, 7 wt % or more, or 10 wt % or more, and 30 wt % or less, 25 wt % or less, 20 wt % or less, or 15 wt % or less, and, relative to the entire liquid composition, are 0.1 to 30 wt %, 0.5 to 30 wt %, 1 to 30 wt %, 3 to 30 wt %, 5 to 30 wt %, 7 to 30 wt %, 10 to 30 wt %, 0.1 to 25 wt %, 0.5 to 25 wt %, 1 to 25 wt %, 3 to 25 wt %, 5 to 25 wt %, 7 to 25 wt %, 10 to 25 wt %, 0.1 to 20 wt %, 0.5 to 20 wt %, 1 to 20 wt %, 3 to 20 wt %, 5 to 20 wt %, 7 to 20 wt %, 10 to 20 wt %, 0.1 to 15 wt %, 0.5 to 15 wt %, 1 to 15 wt %, 3 to 15 wt %, 5 to 15 wt %, 7 to 15 wt %, or 10 to 15 wt %. Within this range, the redispersibility characteristics are excellent when the metal compound particles contained in the liquid composition of the present invention agglomerate and precipitate.

### (B) Metal Compound Particles

The term “metal compound particles” in the present invention refers to organic or inorganic compounds containing metal atoms in the form of particles. The metal compound particles of the present invention are not particularly limited, and examples thereof include zinc oxide particles, magnesium titanate particles, calcium cerium phosphate particles, mica titanium particles, barium sulfate particles, titanium oxide particles, cerium oxide particles, zirconium oxide particles, iron oxide particles, and bismuth oxychloride particles, and these can be used alone or in combination of two or more thereof.

The average particle diameter (before agglomeration) of the metal compound particles used in the present invention is, for example, 0.1 μm or more, 0.5 μm or more, 1 μm or more, 2 μm or more, or 3 μm or more, and 300 μm or less, 250 μm or less, 200 μm or less, 150 μm or less, 100 μm or less, 80 μm or less, 50 μm or less, 30 μm or less, 20 μm or less, or 10 μm or less, and the range of the average particle diameter is 0.1 to 200 μm, 0.1 to 150 μm, 0.1 to 100 μm, 0.1 μm to 50 μm, 0.1 to 20 μm, 0.1 to 10 μm, 2 to 200 μm, 2 to 150 μm, 2 to 100 μm, 2 to 50 μm, 2 to 20 μm, 2 to 10 μm, 3 to 200 μm, 3 to 150 μm, 3 to 100 μm, 3 to 50 μm, 3 to 20 μm, or 3 to 10 μm. The average particle diameter of the metal compound used in the present invention is preferably greater than the average particle diameter of the C₅₋₁₀ alkyl tri-C₁₋₃ alkoxysilane-treated zinc oxide particles and, for example, greater than the average particle diameter of the C₅₋₁₀ alkyl tri-C₁₋₃ alkoxysilane-treated zinc oxide particles by 10 times or more, 20 times or more, 30 times or more, 40 times or more, 50 times or more, 80 times or more, 100 times or more, 150 times or more, or 200 times or more. Within this range, the redispersibility characteristics are excellent when the metal compound particles contained in the liquid composition of the present invention agglomerate and precipitate.

The metal compound particles of the present invention have a specific gravity of 3 or more, 3.5 or more, 4 or more, 4.5 or more, or 5 or more, and 20 or less, 15 or less, 10 or less, 9 or less, 8 or less, 7 or less, or 6 or less, and, for example, have a specific gravity in the range of 3 to 20, 3 to 15, 3 to 10, 3 to 9, 3 to 8, 3 to 7, 3 to 6, 4 to 20, 4 to 15, 4 to 10, 4 to 9, 4 to 8, 4 to 7, 4 to 6, 5 to 20, 5 to 15, 5 to 10, 5 to 9, 5 to 8, 5 to 7, or 5 to 6.

The metal compound particles used in the present invention are, relative to the entire liquid composition, 0.1 wt % or more, 0.2 wt % or more, 0.5 wt % or more, 1.0 wt % or more, 3 wt % or more, or 5 wt % or more, and 25 wt % or less, 20 wt % or less, 15 wt % or less, or 10 wt % or less, and are, relative to the entire liquid composition, 0.1 to 25 wt %, 0.2 to 25 wt %, 0.5 to 25 wt %, 1 to 25 wt %, 3 to 25 wt %, 5 to 25 wt %, 0.1 to 20 wt %, 0.2 to 20 wt %, 0.5 to 20 wt %, 1 to 20 wt %, 3 to 20 wt %, 5 to 20 wt %, 0.1 to 15 wt %, 0.2 to 15 wt %, 0.5 to 15 wt %, 1 to 15 wt %, 3 to 15 wt %, 5 to 15 wt %, 0.1 to 10 wt %, 0.2 to 10 wt %, 0.5 to 10 wt %, 1 to 10 wt %, 3 to 10 wt %, or 5 to 10 wt %. Within this range, the redispersibility characteristics are excellent when the metal compound particles contained in the liquid composition of the present invention agglomerate and precipitate.

The shape of the metal compound particles of the present invention is not particularly limited, and may be any shape such as spherical, needle-like, or plate-like. In the present invention, the “average particle diameter” is a value determined by measurement using, for example, an image analysis device (Luzex IIIU, manufactured by NIRECO), when the particles are not spherical.

The metal compound particles of the present invention may be surface-treated. Examples of surface treatments include silane compound treatment, silicone compound treatment, fluorine-modified silicone compound treatment, fluorine compound treatment, higher fatty acid (stearic acid, etc.) treatment, higher alcohol treatment, fatty acid ester treatment, metal soap treatment, amino acid treatment, and alkyl phosphate treatment.

The metal compound particles of the present invention may be a phosphor. Examples of phosphors include zinc oxide phosphors, magnesium titanate phosphors, and calcium cerium phosphate phosphors, and the liquid composition of the present invention may contain one or more of these phosphors. The phosphor of the metal compound particles can be synthesized by, for example, the method described in Japanese Unexamined Patent Publication (Kokai) No. 2019-167330.

The C₅₋₁₀ alkyl tri-C₁₋₃ alkoxysilane-treated zinc oxide particles of the present invention increase the viscosity of the liquid composition. In order to improve the redispersibility of the metal compound particles, the viscosity of the liquid composition of the present invention is, for example, 500 mPa·s or more, 800 mPa·s or more, 1000 mPa·s or more, 1100 mPa·s or more, 1200 mPa·s or more, 1500 mPa·s or more, 2000 mPa·s or more, 2500 mPa·s or more, or 3000 mPa·s or more, and 10000 mPa·s or less, 9000 mPa·s or less, 8000 mPa·s or less, 7000 mPa·s or less, 6000 mPa·s or less, 5000 mPa·s or less, or 4000 mPa·s or less, and the range of viscosity of the liquid composition of the present invention is 800 to 10000 mPa·s, 1000 to 10000 mPa·s, 1100 to 10000 mPa·s, 1200 to 10000 mPa·s, 1500 to 10000 mPa·s, 2,000 to 10,000 mPa·s, 2,500 to 10,000 mPa·s, or 3,000 to 10,000 mPa·s.

Though the metal compound particles contained in the liquid composition of the present invention precipitate and form aggregates when stored or allowed to stand, by including the C₅₋₁₀ alkyl tri-C₁₋₃ alkoxysilane-treated zinc oxide particles, the redispersibility of the metal compound particles is improved. Though the reason why the C₅₋₁₀ alkyl tri-C₁₋₃ alkoxysilane-treated zinc oxide particles of the present invention exhibit an excellent effect is not clear, it is possible that they precipitate together with the metal compound particles and surround the metal compound particles, thereby preventing the metal compound particles from agglomerating with each other. It is also possible that the C₅₋₁₀ alkyl tri-C₁₋₃ alkoxysilane-treated zinc oxide particles form a structure to increase the static viscosity of the entire liquid composition and prevent the metal compound particles from agglomerating with each other. In any case, the C₅₋₁₀ alkyl tri-C₁₋₃ alkoxysilane-treated zinc oxide particles enhance redispersibility regardless of the type of the metal compound of the present invention and are thus versatile.

The liquid composition of the present invention has metal compound particles that are redispersible, and the precipitated/agglomerated metal compound particles can be redispersed by stirring (or rotation) only 1, 2, 3, 4, 5, 6, 7, 8, 9, or 10 times or fewer.

### Dispersant

The liquid composition of the present invention may contain a dispersant. As used herein, “dispersant” refers to a substance that can adsorb onto particles (powder) dispersed in an aqueous or oily phase and thereby uniformly disperse the particles in an aqueous or oily medium. Examples of preferred dispersants in the liquid compositions of the present invention include PEG-10 dimethicone, bisbutyl dimethicone polyglyceryl-3, PEG-polydimethylpolysiloxane ethyl dimethicone, lauryl PEG-polydimethylpolysiloxane ethyl dimethicone, cetyl PEG/PPG-10/dimethicone, isostearic acid, polyglyceryl-2 diisostearate, carboxydecyltrisiloxane, PEG-12 dimethicone, or polyoxyethylene sorbitan monostearate, or combinations of two or more thereof.

The content of the dispersant in the liquid composition of the present invention is, relative to the entire liquid composition, 0.01 wt % or more, preferably 0.05 mass % or more, more preferably 0.1 wt % or more, and further preferably 0.2 wt % or more, and is 20 wt % or less, preferably 15 wt % or less, more preferably 10 wt % or less, and further preferably 5 wt % or less, and is, relative to the entire liquid composition, 0.01 to 99.99 wt %, 0.1 to 99.9 wt %, 0.05 to 50 wt %, 0.1 to 40 wt %, 0.1 to 30 wt %, 0.1 to 20 wt %, 0.1 to 10 wt %, 0.1 to 5 wt %, 0.2 to 40 wt %, 0.2 to 30 wt %, 0.2 to 20 wt %, 0.2 to 10 wt %, 0.2 to 5 wt %, 0.4 to 40 wt %, 0.4 to 30 wt %, 0.4 to 20 wt %, 0.4 to 10 wt %, or 0.4 to 5 wt % %.

### Oil Component

The liquid composition of the present invention may contain an oil component. As used herein, “oil component” refers to a hydrophobic substance that phase-separates from water, which is a component of the liquid composition of the present invention. Oil components which can be used in the present invention are not particularly limited, and include, for example, at least one or more of a hydrocarbon oil, ester oil, silicone oil, liquid oils and fats, solid oils and fats, and higher alcohol.

Examples of hydrocarbon oils include liquid paraffin, tetraisobutane, hydrogenated polydecene, olefin oligomers, isododecane, isohexadecane, squalane, and hydrogenated polyisobutene.

Examples of ester oils include alkyl benzoates (for example, (C₁₂₋₁₅) alkyl benzoates), diisopropyl sebacate, octyl palmitate, cetyl isooctanoate (cetyl 2-ethylhexanoate), triethylhexanoin, neopentyl glycol dicaprate, triisostearin, diisostearyl malate, PPG-3 dipivalate, di-2-ethylhexyl succinate, 2-ethylhexyl 2-ethylhexanoate, polyglyceryl-6 octacaprylate, and tri(caprylic/capric) glyceryl.

Examples of silicone oils include dimethicone, amino-modified polysiloxane, polyether-modified polysiloxane, alkyl-modified polysiloxane, and fluorine-modified polysiloxane.

Examples of liquid oils and fats include avocado oil, camellia oil, macadamia nut oil, mink oil, olive oil, castor oil, jojoba oil, triglycerin, and glycerin trioctanoate.

Examples of solid oils and fats include coconut oil, hydrogenated coconut oil, palm oil, beef tallow, mutton tallow, Japan wax, and hydrogenated castor oil.

Examples of higher alcohols include isostearyl alcohol, oleyl alcohol, and a copolymer of butylene glycol and propylene glycol (for example, a PBG/PPG-9/1 copolymer).

The content of the total oil component which can be contained in the liquid composition of the present invention is, relative to the entire liquid composition, 5 wt % or more, preferably 10 wt % or more, more preferably 12 wt % or more, and further preferably 15 wt % or more.

### Optional Components

The liquid composition of the present invention can contain various components as appropriate within a range that does not impact the effects of the present invention. Examples of the various components include additives which can be normally added to cosmetics, such as clay minerals (dimethyl distearyl ammonium hectorite, etc.), powders other than the metal compound particles (polymethyl methacrylate, crosslinked silicone/network silicone block copolymers, silica, hydrophobized talc (dimethicone-treated talc, etc.), corn starch, and hydrophobized polyurethane), coating agents (trimethylsiloxysilicate, dimethicone, etc.), transdermal absorption inhibitors (polypropylene glycol(17), etc.), oil phase thickeners (dimethyldistearylammonium hectorite, dextrin palmitate, sucrose triacetate tetrastearate, etc.), ultraviolet absorbers (octocrylene, polysilicone-15, bisethylhexyloxy phenolmethoxy phenyltriazine, diethylaminohydroxy benzoylhexylbenzoate, ethylhexyl salicylate, etc.), UV scattering agents (stearic acid/Al hydroxide-treated titanium oxide, hydrogen dimethicone/Al hydroxide-treated titanium oxide, silica/dimethicone/hydrogen dimethicone-treated titanium oxide, dimethicone-treated zinc oxide. silica/dimethicone-treated zinc oxide, dextrin palmitate-treated zinc oxide, and hydrogen dimethicone-treated zinc oxide) other than the C₅₋₁₀ alkyl tri-C₁₋₃ alkoxysilane-treated zinc oxide particles, chelating agents (sodium edetate hydrate, etc.), fragrances, humectants (glycerin, etc.). preservatives, anionic surfactants, cationic surfactants, amphoteric surfactants, nonionic surfactants, water-soluble polymers, film-forming agents such as siliconized polysaccharides, metal ion sequestering agents, lower alcohols (ethyl alcohol, etc.), polyhydric alcohols, various extracts, sugars, amino acids, organic amines, polymer emulsions, pH adjusters, skin nutrients, vitamins other than vitamin B2, water-soluble agents applicable to pharmaceuticals, quasi-drugs, and cosmetics, antioxidants, buffers, propellants, organic powders, pigments, dyes, coloring agents, water, acid components, and alkali components. These optional components can be appropriately blended in an oil phase and an aqueous phase.

One aspect of the liquid composition of the present invention is directed to a water-in-oil composition. The water-in-oil composition of the present invention can be produced according to conventional production methods.

Specifically, the liquid composition of the present embodiment is obtained by the following procedure. An oil-like component is mixed to prepare an oil phase, and a water-soluble component is mixed to prepare an aqueous phase. The powder of the C₅₋₁₀ alkyl tri-C₁₋₃ alkoxysilane-treated zinc oxide particles and the powder of metal compound particles can be dispersed in the aqueous phase or the oil phase, but it is preferable to disperse them in the same phase. A water-in-oil composition is obtained by adding the water phase to the oil phase and stirring.

The composition of the present invention includes liquid compositions used for makeup bases and sunscreen cosmetics such as sunscreen creams. Examples of the dosage form include a milky lotion.

### Redispersion Facilitator for Metal Compound Particles

One aspect of the present invention is directed to C₅₋₁₀ alkyl tri-C₁₋₃ alkoxysilane-treated zinc oxide particles as a redispersion facilitator for metal compound particles. In the present invention, the term “redispersion facilitator” refers to a compound which improves the redispersibility of metal compound particles which have precipitated and agglomerated in a liquid composition, and facilitates the redispersion of the metal compound particles. The C₅₋₁₀ alkyl tri-C₁₋₃ alkoxysilane-treated zinc oxide particles functioning as a redispersion agent for metal compound particles in a liquid composition can be confirmed by, for example, reducing the number of repetitions of stirring (or rotation) and the stirring (or rotation) time required for redispersion of the metal compound particles in accordance with the method using a stirring ball described in the Examples below:

By way of example, but not limited thereto, according to a method using a stirring ball, it is possible to confirm that C₅₋₁₀ alkyl tri-C₁₋₃ alkoxysilane-treated zinc oxide particles are a redispersion facilitator for metal compound particles. A stirring ball is placed into the liquid composition containing the C₅₋₁₀ alkyl tri-C₁₋₃ alkoxysilane-treated zinc oxide particles and the metal compound particles, and the liquid composition containing the stirring ball is allowed to stand or centrifuged to generate aggregates of the precipitated metal compound particles. Thereafter, the mixture is stirred (or rotated) manually or using a rotator or the like, and the number of repetitions of stirring (or rotation) and the time required for redispersion of the aggregates are measured. It can be confirmed that the measured values for the liquid composition containing the C₅₋₁₀ alkyl tri-C₁₋₃ alkoxysilane-treated zinc oxide particles are less than the measured values for liquid compositions which do not contain the C₅₋₁₀ alkyl tri-C₁₋₃ alkoxysilane-treated zinc oxide particles.

When added to a liquid composition in the method using a stirring ball, the C₅₋₁₀ alkyl tri-C₁₋₃ alkoxysilane-treated zinc oxide particles, which are the redispersion facilitator for metal compound particles of the present invention, can reduce the number of repetitions of stirring (or rotations) required for redispersion of the metal compound particles by 1, 5, 10, 15, 20, 30, 50, 70, 100, 150 or 200 times or more, whereby the number of repetitions of stirring (or rotation) required for redispersion of the metal compound particles can be reduced to ½, ⅓, ¼, ⅕, 1/10 or 1/20, and the metal compound particles contained in the liquid composition can be redispersed by stirring (or rotation) only 1, 2, 3, 4, 5, 6, 7, 8, 9, or 10 times or fewer.

The C₅₋₁₀ alkyl tri-C₁₋₃ alkoxysilane of the C₅₋₁₀ alkyl tri-C₁₋₃ alkoxysilane-treated zinc oxide particles, which are the redispersion facilitator for metal compound particles of the present invention, is a silane compound having an alkyl group having 5 to 10 carbon atoms, and 3 alkoxy groups having 1 to 3 carbon atoms, and has reactivity with a zinc oxide compound. These silane compounds are silane compounds represented by the following general formula (1).

RSiX₃ (1)

where R represents an alkyl group having 5 to 10 carbon atoms (which may be linear or branched), and each X independently represents an alkoxy group having 1 to 3 carbon atoms.

In general formula (1) above, the alkyl group represented by R is an alkyl group having 5 to 10 carbon atoms, and examples thereof include a pentyl group, hexyl group, heptyl group, octyl group, nonyl group, and decyl group, which may be a linear or branched chain. Examples of the alkoxy group represented by X in general formula (1) above include alkoxy groups having 1 to 3 carbon atoms such as a methoxy group, ethoxy group, propoxy group, and isopropoxy group.

Specific examples of silane compounds pentyltrimethoxysilane, hexyltrimethoxysilane, heptyltrimethoxysilane, octyltrimethoxysilane, nonyltrimethoxysilane, decyltrimethoxysilane, pentyltriethoxysilane, hexyltriethoxysilane, heptyltriethoxysilane, octyltriethoxysilane, nonyltriethoxysilane, decyltriethoxysilane, pentyltripropoxysilane, hexyltripropoxysilane, heptyltripropoxysilane, octyltripropoxysilane, nonyltripropoxysilane, decyltripropoxysilane, pentyltriisopropoxysilane, hexyltriisopropoxysilane, heptyltriisopropoxysilane, octyltriisopropoxysilane, nonyltriisopropoxysilane, and decyltriisopropoxysilane. Among these, octyltriethoxysilane and octyltrimethoxysilane are particularly preferred. The silane compound is characterized by easy uniform treatment, easy supply, and inexpensive cost, and zinc oxide particles surface-treated with these compounds have excellent redispersibility characteristics of the metal compound particles contained in the liquid composition of the present invention.

Examples of the method for treating zinc oxide particles with the silane compound include a method of a chemical reaction of the silane compound via reactive groups (alkoxy group, etc.) on the surface of the zinc oxide particle powder, such as a method, in which the silane compound and the zinc oxide particles (powder) are mixed in an organic solvent such as n-hexane, cyclohexane, or a lower alcohol, and optionally, the powder is pulverized, and thereafter, the organic solvent is removed by heating or pressure reduction, and heat treatment is carried out, preferably at 80 to 250° C.

Another example is the method described in Japanese Unexamined Patent Publication (Kokai) No. 2007-326902, in which a zinc oxide particle powder is coated with a specific polysiloxane compound and the surface thereof is then treated with the silane compound or silazane compound in water.

As another example, there is a method in which a zinc oxide particle powder is coated thereon in advance with an inorganic oxide such as silica, alumina, zirconia, titanium oxide, iron oxide, or cerium oxide, and the inorganic oxide-treated zinc oxide particle powder is then surface-coated with the silane compound. Examples of a method for producing the inorganic oxide-treated zinc oxide particle powder include conventionally-known treatment methods such as wet treatment methods using a solvent and mechanochemical methods. An example is the method described in WO 98/17730, in which a zinc oxide particle powder is coated thereon with a silicone compound and then fired to obtain a silica-coated zinc oxide particle powder.

The coating amount of silane compound on the zinc oxide particle powder is preferably 3 to 15 mass %, and more preferably 4 to 10 mass %, based on the total amount of the zinc oxide particle powder used. Within this range, the surface of the zinc oxide particle powder is uniformly coated with the silane compound, whereby there is no agglomeration or precipitation of the silane compound on the surface of the zinc oxide particle powder.

The C₅₋₁₀ alkyl tri-C₁₋₃ alkoxysilane-treated zinc oxide particles, which are the redispersion facilitator for metal compound particles of the present invention, preferably have an average particle diameter of 200 nm or less, 100 nm or less, 50 nm or less, or 40 nm or less, and more preferably 50 nm or less, 40 nm or less or 35 nm or less. The average particle diameter of the C₅₋₁₀ alkyl tri-C₁₋₃ alkoxysilane-treated zinc oxide particles, which are the redispersion facilitator for metal compound particles, is preferably less than the average particle diameter of the metal compound, and is, for example, 1/10 or less, 1/20 or less, 1/30 or less, 1/40 or less, 1/50 or less, 1/90 or less, 1/100 or less, 1/150 or less, or 1/200 or less of the average particle diameter of the metal compound (before agglomeration). Within this range, the redispersibility characteristics are excellent when the metal compound particles contained in the liquid composition of the present invention agglomerate and precipitate.

The C₅₋₁₀ alkyl tri-C₁₋₃ alkoxysilane-treated zinc oxide particles, which are the redispersion facilitator for metal compound particles of the present invention, can be used at a concentration of, relative to the entirety of the liquid composition, 0.1 wt % or more, 0.5 wt % or more, 1.0 wt % or more, 3 wt % or more, 5 wt % or more, 7 wt % or more, or 10 wt % or more, and 30 wt % or less, 25 wt % or less, 20 wt % or less, or 15 wt % or less, and relative to the entire liquid composition, 0.1 to 30 wt %, 0.5 to 30 wt %, 1 to 30 wt %, 3 to 30 wt %, 5 to 30 wt %, 7 to 30 wt %, 10 to 30 wt %, 0.1 to 25 wt %, 0.5 to 25 wt %, 1 to 25 wt %, 3 to 25 wt %, 5 to 25 wt %, 7 to 25 wt %, 10 to 25 wt %, 0.1 to 20 wt %, 0.5 to 20 wt %, 1 to 20 wt %, 3 to 20 wt %, 5 to 20 wt %, 7 to 20 wt %, 10 to 20 wt %, 0.1 to 15 wt %, 0.5 to 15 wt %, 1 to 15 wt %, 3 to 15 wt %, 5 to 15 wt %, 7 to 15 wt %, or 10 to 15 wt %. Within this range, the redispersibility characteristics are excellent when the metal compound particles contained in the liquid composition of the present invention agglomerate and precipitate.

Metal compound particles to which the redispersion facilitator of the present invention can be applied include, but are not limited to, zinc oxide particles, magnesium titanate particles, calcium cerium phosphate particles, titanium mica particles, barium sulfate particles, titanium oxide particles, cerium oxide particles, zirconium oxide particles, iron oxide particles, and these can be used alone or in combination of two or more thereof.

The average particle diameter (before agglomeration) of the metal compound particles to which the redispersion facilitator of the present invention is applied is, for example, 0.1 μm or more, 0.5 μm or more, 1 μm or more, 2 μm or more, or 3 μm or more, and 300 μm or less, 250 μm or less, 200 μm or less, 150 um or less, 100 μm or less, 80 μm or less, 50 μm or less, 30 μm or less, 20 μm or less, or 10 μm or less, and the range of the average particle diameter is 0.1 to 200 μm, 0.1 to 150 μm, 0.1 to 100 μm, 0.1 μm to 50 μm, 0.1 to 20 μm, 0.1 to 10 μm, 2 to 200 μm, 2 to 150 μm, 2 to 100 μm, 2 to 50 μm, 2 to 20 μm, 2 to 10 μm, 3 to 200 μm, 3 to 150 μm, 3 to 100 μm, 3 to 50 μm, 3 to 20 μm, or 3 to 10 μm. The average particle diameter of the metal compound to which the redispersion facilitator of the present invention is applied is preferably greater than the average particle diameter of the C₅₋₁₀ alkyl tri-C₁₋₃ alkoxysilane-treated zinc oxide particles and, for example, greater than the average particle diameter of the C₅₋₁₀ alkyl tri-C₁₋₃ alkoxysilane-treated zinc oxide particles by 10 times or more, 20 times or more, 30 times or more, 40 times or more, 50 times or more, 80 times or more, 100 times or more, 150 times or more, or 200 times or more. Within this range, the redispersibility characteristics are excellent when the metal compound particles agglomerate and precipitate.

The metal compound particles to which the redispersion facilitator of the present invention is applied have a specific gravity of 3 or more, 3.5 or more, 4 or more, 4.5 or more, or 5 or more, and 20 or less, 15 or less, 10 or less, 9 or less, 8 or less, or 7 or less, and, for example, have a specific gravity in the range of 3 to 20, 3 to 15, 3 to 10, 3 to 9, 3 to 8, 3 to 7, 4 to 20, 4 to 15, 4 to 10, 4 to 9, 4 to 8, 4 to 7, 5 to 20, 5 to 15, 5 to 10, 5 to 9, 5 to 8, or 5 to 7.

The shape of the metal compound particles to which the redispersion facilitator of the present invention is applied is not particularly limited, and may be spherical, needle-like, or plate-like. The “average particle diameter” of the present invention is, for example, a value determined by measurement with an image analyzer (Luzex IIIU, manufactured by Nireco) when the particles are not spherical, and is determined as a number-average circle equivalent diameter.

The metal compound particles to which the redispersion facilitator of the present invention is applied may be surface-treated. Examples of surface treatments include silane compound treatment (octyltriethoxyran, etc.), silicone compound treatment, fluorine-modified silicone compound treatment, fluorine compound treatment, higher fatty acid (stearic acid, etc.) treatment, higher alcohol treatment, fatty acid ester treatment, metal soap treatment, amino acid treatment, and alkyl phosphate treatment.

The metal compound particles to which the redispersion facilitator of the present invention is applied may be a phosphor. Examples of phosphors include zinc oxide phosphors, magnesium titanate phosphors, and calcium cerium phosphate phosphors, and the liquid composition of the present invention may contain one or more of these phosphors. The phosphor of the metal compound particles can be synthesized by, for example, the method described in Japanese Unexamined Patent Publication (Kokai) No. 2019-167330.

Though the metal compound particles, to which the redispersion facilitator of the present invention is applied, precipitate and form aggregates when stored or allowed to stand, the C₅₋₁₀ alkyl tri-C₁₋₃ alkoxysilane-treated zinc oxide particles, which are a redispersion facilitator, improve redispersibility: Though it is unclear why the C₅₋₁₀ alkyl tri-C₁₋₃ alkoxysilane-treated zinc oxide particles, which are the redispersion facilitator of the present invention, exhibit excellent effects, it is possible that they precipitate together with the metal compound particles and surround the metal compound particles, thereby preventing the metal compound particles from agglomerating with each other. It is also possible that the C₅₋₁₀ alkyl tri-C₁₋₃ alkoxysilane-treated zinc oxide particles, which are a redispersion facilitator, form a structure to increase the static viscosity of the entire liquid composition and prevent the metal compound particles from agglomerating with each other. In any case, the C₅₋₁₀ alkyl tri-C₁₋₃ alkoxysilane-treated zinc oxide particles, which are a redispersion facilitator, enhance redispersibility regardless of the type of the metal compound of the present invention and are thus versatile.

One aspect of the present invention is directed to the use of the C₅₋₁₀ alkyl tri-C₁₋₃ alkoxysilane-treated zinc oxide particles as a redispersion facilitator for metal compound particles, for the redispersion of the metal compound particles precipitated in a composition. By adding the C₅₋₁₀ alkyl tri-C₁₋₃ alkoxysilane-treated zinc oxide particles to a liquid composition, the metal compound particles contained in the liquid composition can be redispersed by stirring (or rotation) only 1, 2, 3, 4, 5, 6, 7, 8, 9, or 10 times.

One aspect of the present invention is directed to the use of C₅₋₁₀ alkyl tri-C₁₋₃ alkoxysilane-treated zinc oxide particles as a redispersion facilitator for metal compound particles. In order to easily redisperse the metal compound particles which have precipitated and agglomerated in the liquid composition, the C₅₋₁₀ alkyl tri-C₁₋₃ alkoxysilane-treated zinc oxide particles can be added as a component of the liquid composition.

### EXAMPLES

The present invention will be described in further detail with reference to the following Examples. Note that the present invention is not limited thereto.

### Example 1: Effects of Octyltriethoxysilane-Treated Zinc Oxide

### 1-1) Redispersibility Test Using Stirring Ball

35 mL of the liquid composition to be tested was transferred into a centrifugation bottle (50 mL polypropylene Falcon conical tube: manufactured by Corning), one stirring ball (composed of steel, diameter: 5.56 mm) was added thereto, centrifuged at 740 rpm (110×g) for 60 minutes at room temperature to precipitate and agglomerate the metal compound particles, whereby the storage state of the liquid composition for a certain period of time was reproduced (centrifuge: CF7D2: manufactured by Himac). Thereafter, the centrifugation bottle was shaken up and down by hand, and the redispersibility was evaluated by counting the number of repetitions of stirring when the stirring ball produced sound.

### 1-2) Facilitation of Redispersion by Octyltriethoxysilane-Treated Zinc Oxide

The zinc oxide phosphor contained in the liquid composition sometimes precipitates and forms aggregates during storage. The effects of the following six types of compounds, which are known as ultraviolet scattering agents, on the redispersibility of a zinc oxide phosphor in the liquid composition were investigated using the “1-1) Redispersibility Test Using Stirring Ball” described above. The component compositions of the tested liquid compositions are as shown in Table 1, and they were produced according to a conventional manufacturing method. The zinc oxide phosphor used was Lumate G (specific gravity: 5.6, particle size: 3 μm) (Sakai Chemical Industry Co., Ltd.) treated with octyltriethoxysilane:
1) Formulation Example 1: Octyltriethoxysilane-treated zinc oxide (particle size: 25 nm)
2) Formulation Example 2: Octyltriethoxysilane-treated zinc oxide (particle size: 35 nm)
3) Formulation Example 3: Dimethicone-treated zinc oxide (particle size: 25 nm)
4) Formulation Example 4: Silica/dimethicone-treated zinc oxide (particle size: 20 nm)
5) Formulation Example 5: Dextrin palmitate-treated zinc oxide (particle size: 25 nm)
6) Formulation Example 6: Hydrogen dimethicone-treated zinc oxide (particle size: 50 nm)

TABLE 1

| | Form | Form | Form | Form | Form | Form |
|---|---|---|---|---|---|---|
| Composition | Ex 1 | Ex 2 | Ex 3 | Ex 4 | Ex 5 | Ex 6 |
| Water | 9.7 | 9.7 | 9.7 | 9.7 | 9.7 | 9.7 |
| Ethanol | 4 | 4 | 4 | 4 | 4 | 4 |
| Glycerin | 1 | 1 | 1 | 1 | 1 | 1 |
| Trimethylsiloxysilicic acid, dimethicone | 1 | 1 | 1 | 1 | 1 | 1 |
| PEG-9 polydimethylpolysiloxyethyl dimethicone | 1.5 | 1.5 | 1.5 | 1.5 | 1.5 | 1.5 |
| Bisbutyl dimethicone polyglyceryl-3 | 1 | 1 | 1 | 1 | 1 | 1 |
| Polypropylene glycol(17) | 1 | 1 | 1 | 1 | 1 | 1 |
| Dimethyl distearyl ammonium hectorite | 0.3 | 0.3 | 0.3 | 0.3 | 0.3 | 0.3 |
| Dextrin palmitate | 2 | 2 | 2 | 2 | 2 | 2 |
| Sucrose triacetate tetrastearate | 1 | 1 | 1 | 1 | 1 | 1 |
| (C₁₂₋₁₅)alkyl benzoate | 3.7 | 3.7 | 3.7 | 3.7 | 3.7 | 3.7 |
| Diisopropyl sebacate | 10 | 10 | 10 | 10 | 10 | 10 |
| Dimethicone (1.5 cst) | 23 | 23 | 23 | 23 | 23 | 23 |
| Octocrylene | 5 | 5 | 5 | 5 | 5 | 5 |
| Polysilicone-15 | 3 | 3 | 3 | 3 | 3 | 3 |
| Bisethylhexyloxyphenol methoxyphenyltriazine | 2.5 | 2.5 | 2.5 | 2.5 | 2.5 | 2.5 |
| Diethylaminohydroxybenzoyl hexyl benzoate | 2 | 2 | 2 | 2 | 2 | 2 |
| Ethylhexyl salicylate | 5 | 5 | 5 | 5 | 5 | 5 |
| Stearic acid/Al hydroxide-treated titanium oxide (particle | 2 | 2 | 2 | 2 | 2 | 2 |
| size: 15 nm) | | | | | | |
| Octyltriethoxysilane-treated zinc oxide (particle size: 25 | 11 | | | | | |
| nm) | | | | | | |
| Octyltriethoxysilane-treated zinc oxide (particle size: 35 | | 11 | | | | |
| nm) | | | | | | |
| Dimethicone-treated zinc oxide (particle size: 25 nm) | | | 11 | | | |
| Silica/dimethicone-treated zinc oxide (particle size: 20 nm) | | | | 11 | | |
| Dextrin palmitate-treated zinc oxide (particle size 25 nm) | | | | | 11 | |
| Hydrogen dimethicone-treated zinc oxide (particle size 50 | | | | | | 11 |
| nm) | | | | | | |
| Octyltriethoxysilane-treated zinc oxide phosphor (specific | 5.2 | 5.2 | 5.2 | 5.2 | 5.2 | 5.2 |
| gravity: 5.6, particle size: 3 μm) | | | | | | |
| Dimethicone-treated talc | 4 | 4 | 4 | 4 | 4 | 4 |
| Spherical silica (particle size: 5 μm, porous) | 1 | 1 | 1 | 1 | 1 | 1 |
| EDTA · 3Na—2H₂O | 0.1 | 0.1 | 0.1 | 0.1 | 0.1 | 0.1 |
| Number of repetitions of stirring for redispersion | 6 | 7 | >200 | 60 | >200 | 30 |
| Viscosity (mPa · s) | 4240 | 3820 | 2130 | 2920 | 2470 | 2670 |

As shown in Table 1, the octyltriethoxysilane-treated zinc oxide particles significantly improved the redispersibility of the zinc oxide phosphor as compared to other UV scattering agents. Relative to the zinc oxide phosphor having a particle diameter (average particle diameter) of 3 μm, octyltriethoxysilane-treated zinc oxide particles having a particle diameter of 25 and 35 nm had improved redispersibility. When the viscosities of the Formulation Examples were examined with a viscosity meter (VISCOMETER TVB-15; manufactured by TOKI SANGYO), it was found that the octyltriethoxysilane-treated zinc oxide increased the viscosity as compared to other UV scattering agents. From this, it is suggested that the octyltriethoxysilane-treated zinc oxide particles may form a structure to increase the static viscosity of the entire liquid composition, thereby suppressing the precipitation and agglomeration of the zinc oxide phosphor, which is a metal compound.

### Example 2: Effect on Various Metal Compound Particles

Next, the effect of octyltriethoxysilane-treated zinc oxide on the redispersibility of the following four types of metal compound particles other than the zinc oxide phosphor was examined by the “1-1) Redispersibility Test Using Stirring Ball” described above. The component compositions of the tested liquid compositions are as shown in Table 2, and were produced according to a conventional manufacturing method:
1) Formulation Examples 7 and 8: stearic acid-treated zinc oxide phosphor (specific gravity: 5.6, particle size: 3 μm)
2) Formulation Examples 9 and 10: Magnesium titanate phosphor (specific gravity: 4.5, particle size: 3 to 4 μm)
3) Formulation Examples 11 and 12: Barium sulfate (specific gravity: 4.4, particle size: 10 to 20 μm)
4) Formulation Examples 13 and 14: Mica titanium (specific gravity: 3.1, particle size: 22 to 74 μm)

TABLE 2

| | Form | Form | Form | Form | Form | Form | Form | Form |
|---|---|---|---|---|---|---|---|---|
| Composition | Ex 7 | Ex 8 | Ex 9 | Ex 10 | Ex 11 | Ex 12 | Ex 13 | Ex 14 |
| Water | 9.9 | 9.9 | 9.9 | 9.9 | 9.9 | 9.9 | 9.9 | 9.9 |
| Ethanol | 4 | 4 | 4 | 4 | 4 | 4 | 4 | 4 |
| Glycerin | 1 | 1 | 1 | 1 | 1 | 1 | 1 | 1 |
| Trimethylsiloxysilicic acid, dimethicone | 1 | 1 | 1 | 1 | 1 | 1 | 1 | 1 |
| PEG-9 polydimethylpolysiloxyethyl dimethicone | 1.5 | 1.5 | 1.5 | 1.5 | 1.5 | 1.5 | 1.5 | 1.5 |
| Bisbutyl dimethicone polyglyceryl-3 | 1 | 1 | 1 | 1 | 1 | 1 | 1 | 1 |
| Polypropylene glycol(17) | 1 | 1 | 1 | 1 | 1 | 1 | 1 | 1 |
| Dimethyl distearyl ammonium hectorite | 0.3 | 0.3 | 0.3 | 0.3 | 0.3 | 0.3 | 0.3 | 0.3 |
| Dextrin palmitate | 2 | 2 | 2 | 2 | 2 | 2 | 2 | 2 |
| Sucrose triacetate tetrastearate | 1 | 1 | 1 | 1 | 1 | 1 | 1 | 1 |
| (C₁₂₋₁₅)alkyl benzoate | 3.7 | 3.7 | 3.7 | 3.7 | 3.7 | 3.7 | 3.7 | 3.7 |
| Diisopropyl sebacate | 10 | 10 | 10 | 10 | 10 | 10 | 10 | 10 |
| Dimethicone (1.5 cst) | 23 | 23 | 23 | 23 | 23 | 23 | 23 | 23 |
| Octocrylene | 5 | 5 | 5 | 5 | 5 | 5 | 5 | 5 |
| Polysilicone-15 | 3 | 3 | 3 | 3 | 3 | 3 | 3 | 3 |
| Bisethylhexyloxyphenol methoxyphenyltriazine | 2.5 | 2.5 | 2.5 | 2.5 | 2.5 | 2.5 | 2.5 | 2.5 |
| Diethylaminohydroxybenzoyl hexyl benzoate | 2 | 2 | 2 | 2 | 2 | 2 | 2 | 2 |
| Ethylhexyl salicylate | 5 | 5 | 5 | 5 | 5 | 5 | 5 | 5 |
| Stearic acid/Al hydroxide-treated titanium oxide | 2 | 2 | 2 | 2 | 2 | 2 | 2 | 2 |
| (particle size: 15 nm) | | | | | | | | |
| Dimethicone-treated zinc oxide (particle size: 25 | | 11 | | 11 | | 11 | | 11 |
| nm) | | | | | | | | |
| Octyltriethoxysilane treated zinc oxide (particle | 11 | | 11 | | 11 | | 11 | |
| size 25 nm) | | | | | | | | |
| Stearic acid-treated zinc oxide phosphor (specific | 5 | 5 | | | | | | |
| gravity: 5.6, particle size: 3 μm) | | | | | | | | |
| Magnesium titanate phosphor (specific gravity: | | | 5 | 5 | | | | |
| 4.5, particle size: 3 to 4 μm) | | | | | | | | |
| Barium sulfate (specific gravity: 4.4, particle: size | | | | | 5 | 5 | | |
| 10 to 20 μm) | | | | | | | | |
| Mica titanium (specific gravity: 3.1, particle size: | | | | | | | 5 | 5 |
| 22 to 74 μm) | | | | | | | | |
| Dimethicone-treated talc | 4 | 4 | 4 | 4 | 4 | 4 | 4 | 4 |
| Spherical silica (particle size: 5 μm, porous) | 1 | 1 | 1 | 1 | 1 | 1 | 1 | 1 |
| EDTA · 3Na—2H₂O | 0.1 | 0.1 | 0.1 | 0.1 | 0.1 | 0.1 | 0.1 | 0.1 |
| Number of repetitions of stirring for redispersion | 7.0 | >200 | 8.0 | >100 | 7.0 | 57.0 | 3.0 | 28.0 |
| Viscosity (mPa · s) | 2300 | 1200 | 2170 | 1230 | 2820 | 1160 | 2620 | 1310 |

As shown in Table 2, the octyltriethoxysilane-treated zinc oxide improved the redispersibility of the stearic acid-treated zinc oxide phosphor. Thus, it was found that even when the metal compound particles were surface-treated, the effect of the octyltriethoxysilane-treated zinc oxide was recognized. Likewise, it was found that octyltriethoxysilane-treated zinc oxide improved the redispersibility of magnesium titanate, barium sulfate, and titanium mica, and this effect was brought about regardless of the type of metal compound particles. In each case, the octyltriethoxysilane-treated zinc oxide increased the viscosity of the liquid composition, but at the same level as in Formulation Examples 3 to 6, which resulted in poor redispersibility. Since octyltriethoxysilane-treated zinc oxide is thought to precipitate together with metal compound particles, it is suggested that the octyltriethoxysilane-treated zinc oxide may form a structure to increase the static viscosity of the entire liquid composition, and act directly to improve redispersibility, such as surround the metal compound particles to suppress agglomeration.

From the foregoing, it was found that a liquid composition containing octyltriethoxysilane-treated zinc oxide particles and metal compound particles is excellent in redispersibility of the metal compound particles, and octyltriethoxysilane-treated zinc oxide particles improve the redispersibility of the metal compound particles and are useful as a redispersion facilitator for metal compound particles.

1. A liquid composition, comprising:
(A) C₅₋₁₀ alkyl tri-C₁₋₃ alkoxysilane-treated zinc oxide particles having an average particle diameter of 100 nm or less, and
(B) metal compound particles having a concentration of 0.2 wt % or more, a specific gravity of 3 or more, and an average particle diameter of 1 μm or more.

2. The liquid composition according to claim 1, wherein a concentration of the C₅₋₁₀ alkyl tri-C₁₋₃ alkoxysilane-treated zinc oxide particles is 1 wt % or more.

3. The liquid composition according to claim 1, wherein a viscosity of the liquid composition is 10000 mPa·s or less.

4. The liquid composition according to claim 1, wherein the metal compound particles are one or more powders selected from the group consisting of barium sulfate, titanium mica, zinc oxide, zinc oxide phosphor, magnesium titanate, magnesium titanate phosphor, calcium phosphate, calcium cerium phosphate phosphor, and bismuth oxychloride.

5. The liquid composition according to claim 1, wherein the C₅₋₁₀ alkyl tri-C₁₋₃ alkoxysilane-treated zinc oxide particles are octyltriethoxysilane-treated zinc oxide particles or octyltrimethoxysilane-treated zinc oxide particles.

6. The liquid composition according to claim 1, wherein the metal compound particles, when precipitated, are redispersible.

7. The liquid composition according to claim 1, which is a water-in-oil composition.

8. The liquid composition according to claim 1, which is a sunscreen cosmetic.

9. A redispersion facilitator for metal compound particles which precipitate and agglomerate in a liquid composition, the redispersion facilitator comprising C₅₋₁₀ alkyl tri-C₁₋₃ alkoxysilane-treated zinc oxide particles as an active ingredient.

10. The redispersion facilitator according to claim 9, wherein the metal compound particles are a powder having a specific gravity of 3 or more and an average particle diameter of 1 μm or more.

11. The redispersion facilitator according to claim 9, wherein the metal compound particles are one or more powders selected from the group consisting of barium sulfate, titanium mica, zinc oxide, zinc oxide phosphor, magnesium titanate, magnesium titanate phosphor, calcium phosphate, calcium cerium phosphate phosphor, and bismuth oxychloride.

12. The redispersion facilitator according to claim 9, wherein the C₅₋₁₀ alkyl tri-C₁₋₃ alkoxysilane-treated zinc oxide particles are octyltriethoxysilane-treated zinc oxide particles or octyltrimethoxysilane-treated zinc oxide particles.

13. A method, for redispersion of metal compound particles which precipitate and agglomerate in a liquid composition, comprising adding a redispersion facilitator according to claim 9 to the liquid.
